(19) United States
(12) Patent Application Publication
(10) Pub. No.: US 2024/0268691 A1
(43) Pub. Date: Aug. 15, 2024

(54) OPTOELECTRONIC BIOSENSOR AND METHOD

(71) Applicant: ams-OSRAM International GmbH, Regensburg (DE)

(72) Inventors: Sergey KUDAEV, Regensburg (DE); Jens RICHTER, Hemau (DE); Klaus FLOCK, Regensburg (DE)

(21) Appl. No.: 18/568,392

(22) PCT Filed: Jun. 10, 2022
(86) PCT No.: PCT/EP2022/065806

§ 371 Date: Dec. 8, 2023

(30) Foreign Application Priority Data
Jun. 10, 2021 (DE) 10 2021 115 049.3

(51) Int. Cl. A61B 5/024

(52) U.S. Cl. CPC A61B 5/02427; A61B 2562/0238; A61B 2562/0242

(57) ABSTRACT

An optoelectronic biosensor includes a housing with a transparent support surface. The optoelectronic biosensor also includes an emitter device in a first region of the housing adapted to generate and emit light toward the support surface. The optoelectronic biosensor further includes a detector device in a second region of the housing. The optoelectronic biosensor additionally includes a first optical fiber element between the detector device and the support surface. The first optical fiber element is configured to guide light incident on the support surface at an angle less than a predetermined angle to a normal to the detector device. The predetermined angle depends at least on a difference in refractive indices at an interface between the optical fiber element and a material surrounding the optical light guide element. A material of the first region surrounding the emitter device is the same as the material surrounding the first optical fiber element.

The present application claims priority from the German first application DE 10 2021 115 049.3 of Jun. 10, 2021, the disclosure content of which is hereby incorporated by reference in its entirety.

The present invention relates to an optoelectronic biosensor and a method of manufacturing the same.

### BACKGROUND

Optical biosensors are used to measure light emitted or reflected by a tissue by means of sensors. Depending on the application, the light components can then be used to infer various physiological parameters. For example, photoplethysmography can be performed using a biosensor in which light emitted by the sensor interacts with human tissue. This also results in absorption of light by hemoglobin in deeper tissue layers. The absorption process causes a slow modulation of the reflected light in unison with the heartbeat. A corresponding light detector in the biosensor can detect this modulation in a light signal that has reflected-back components.

However, this modulation is quite weak because most of the light emitted by the biosensor is already reflected back at the surface or through non-perfused tissue layers and other effects without producing a modulation due to strong scattering. Thus, non-modulated light also enters the detector and causes a DC signal there, which cannot contribute any information to the heart rate measurement. This DC component worsens the signal/noise ratio.

Accordingly, there is a need to improve the signal-to-noise ratio and reduce light components that make little or no contribution to the measurement.

### SUMMARY OF THE INVENTION

This need is met by the subject matter of the independent claims. Various embodiments and further measures are the subject of the dependent claims.

The inventors have recognized that in such above measurements, due to the interaction of light with human tissue, the light is scattered or absorbed and reabsorbed. Only a portion of the light propagates back after interacting with the tissue and is detected by a photodetector device of the biosensor. Due to the strong scattering of light by the uppermost and non-perfused tissue layers, the non-modulated portion of a signal detected by the biosensor's photodetector device is relatively large. This non-modulated component is also referred to as the DC component in the following. It forms a signal portion in the detector device that belongs to the non-usable portion and increases the noise from the system.

In subsequent signal processing, special measures must therefore be taken to reduce these DC components of the signal in order to obtain a sufficient signal-to-noise ratio, SNR. The DC components of the signal are composed of an internal crosstalk of light from the light source of the biosensor directly to the detector device of the biosensor, a scattering of light at tissue layers close to the surface and a scattering of light in deeper tissue layers, but without interaction with blood or hemoglobin or any other tissue producing the desired interaction. With the proposed principle, signal components resulting from direct crosstalk or from scattering at the uppermost tissue layers can now be reduced.

To this end, the inventors propose an optoelectronic biosensor comprising a housing having a transparent contact surface. In the application of the optoelectronic biosensor, the transparent contact surface serves to come into contact with skin or other tissue. Similarly, a transparent protective screen may be provided. For example, in an application with the transparent contact surface, the optoelectronic biosensor is applied to the skin for sensing heart rate within tissue layers.

The optoelectronic biosensor further comprises an emitter device in a first region of the housing of the biosensor, the emitter device being configured to generate and emit light toward the support surface. A detector device is provided in a second region of the housing. According to the invention, a first optical fiber element is arranged between the detector device and the supporting surface. This is designed to guide light at an angle smaller than a predetermined angle to a perpendicular to the support surface in the direction of the detector device. The predetermined angle depends at least on a difference of the refractive indices at an interface between the optical fiber element and a material surrounding the optical light guide element.

In the biosensor according to the proposed principle, the inventors exploit the fact that light hitting the contact surface is angle-dependent due to the interaction with the various skin surfaces. It was found that light which strikes the contact surface at a relatively steep angle, i.e. a small angle with respect to a perpendicular, originates primarily from deeper tissue layers. This light component contains, among other things, a particularly high proportion of light which interacted with hemoglobin or blood.

On the other hand, light from the non-perfused tissue layers near the surface strikes the respective contact surface at a particularly large angle to the perpendicular. By means of an appropriately arranged optical light guide element, it is now possible to ensure that light components above a critical angle leave the optical fiber element again, and in particular are not reflected back at the interface between the optical fiber element and the material surrounding the light guide element, but are refracted out.

In other words, light components that strike the contact surface at an angle smaller than a predetermined angle are reflected at the interface between the optical fiber element and the surrounding material and thus enter the detector device. If the angle is larger than the predetermined angle, light components are guided away from the optical detector device according to the proposed principle. It was found that such light components originate mainly from scattered light from tissue layers close to the surface. The signal-to-noise ratio is improved by reducing these DC signal components. The terms “predetermined angle” or “critical angle” are used synonymously here and in the following.

In contrast to conventional products, where the light emitter unit as well as the photodetector have to be placed at a relatively large distance from each other in order to reduce such stray light, with the approach described it is possible to achieve effective suppression of DC signal components due to direct crosstalk even at relatively small distances between emitter and detector. This enables applications characterized by small size and allows such biosensors to be implemented in relatively small spaces in the range of 2 mm to 3 mm as the distance between the detector and emitter. In some aspects, the total area can also be in the range of 2 mm² to 1². Also, the thickness of such a biosensor can be reduced due to the optical fiber element provided above the detector array. Such a biosensor thus allows measurements to be made, for example, on a fingertip, on a limb of a finger, but also in the ear canal or ear or other areas of the body that require a small size of the biosensor for good access.

To produce suitable light guidance, a material of the first optical fiber element comprises a higher refractive index than the material surrounding the first optical light guide element. Due to the different refractive indices in the specified manner, a light coupled into the first optical fiber element below the specified or critical angle is reflected back into the optical light guide when it strikes the interface, and thus enters the detection device. In this context, the terms refractive index and refractive index are used interchangeably.

The given angle depends on the different refractive indices between the material of the first optical fiber element and the material surrounding the optical light guide element. In general it can be said that the larger this refractive index jump is, the smaller the acceptance angle or the larger the critical angle is or should be. Accordingly, the signal-to-noise ratio, which in turn is a function of the predetermined angle, can thus be adjusted by the different materials of the light guide element and the material surrounding the light guide element. In one aspect, the predetermined angle is proportional to an arc sine of a root of the difference of the two respective squared refractive indices.

In a further aspect, at least the second region, i.e., the region in which the detector device is disposed, is filled with the material surrounding the first optical fiber element up to a height of the support surface. This material, like the material of the first optical light guide element, is transparent to the light coupling into the support surface. Suitable possible transparent materials for the first optical fiber element include, for example, silicones or even polycarbonates having a refractive index of n>1.6. However, an epoxy resin or other plastic having a lower refractive index, for example, of n<=1.5, can be used as the material for the material surrounding the optical light guide element. In some aspects, a particular glass that also comprises a high refractive index can also be used for the optical light guide element.

In some aspects, in order to prevent a light component emerging from the optical fiber element from passing back through the housing wall into the detector element and leading to an unusable signal component there, it is provided that a surface of the housing facing the second region is designed to be absorbent for the light emitted by the emitter device. In particular, this can be the housing wall but also a housing bottom. The absorption can be caused, for example, by an additional layer on the housing but also by the material used for the housing. Particularly absorbing particles such as carbon black, carbon and others are suitable here.

In one embodiment of the invention, it is convenient to also surround the emitter unit with a transparent material so that it is protected from external environmental influences. The material surrounding the emitter device may be the same material as the material surrounding the first optical light guide element. If the same materials are used, the cost of manufacturing such a biosensor can be reduced. For example, it is possible to glue or otherwise attach the first optical fiber element to the detector device and then fill the second region and the first region of the housing with the further transparent material.

Another aspect of the proposed principle deals with improving the radiation pattern of the emitter device so that the usable portion of the signal incident on the detector device is further increased. In this regard, therefore, a second optical fiber element may be disposed between the emitter device and the support surface for guiding light. The second optical fiber element may have the same material as the first optical fiber element and thus serve as an optical waveguide for the light emitted from the emitter device.

In addition, the second optical fiber element can also comprise light guide elements, such as lenses or collimators. This makes it possible to emit light in a more directed manner, which increases the penetration depth of the light and also the proportion of the light that penetrates into deeper tissue layers. In this respect, the proportion of light relevant for interaction with blood or hemoglobin can be increased, so that the overall signal-to-noise ratio is further improved.

For a reflection of light components within the first or the second optical light guide element, in addition to the already explained refractive index jump at the interface, the angle of the impingement of the light on the interface is also of importance. Accordingly, in some aspects, it is contemplated that a particular shape of the first optical fiber element or the second optical fiber element may be used to adjust this angle to provide greater flexibility with respect to the various light components impinging on the support surface.

In some aspects, such a change in shape of the optical fiber element is provided by a funnel-shaped or pyramid-shaped configuration. However, other shapes may also be possible. In some aspects, the first or second optical fiber element is configured with a cross-section that tapers toward the support surface. Thus, the light guide element is pyramidal or conical in shape, such that the predefined or critical angle at which total internal reflection occurs increases. This can result in more useful signal entering the detector due to reflections at the interface, but at the same time reducing the entrance window or area of the first or second optical fiber element along the support surface.

In another aspect, the cross-section of the first or second optical fiber element is increased relative to the supporting surface so that a funnel-shaped configuration is realized. This increases the entrance window of the optical light guide elements so that both the useful signal component and a background signal component, i.e., a DC component, are increased. However, the critical angle is reduced so that a larger signal component hitting the interface is refracted away from the detector and into the surrounding material.

Nevertheless, an improved flexibility is achieved in such an embodiment, since the radiation characteristic as well as the reception characteristic of the biosensor can be designed more flexibly by these measures and the shape of the light guide elements. Together with the height of the light guide elements, the distance between emitter and detector device, a large parameter space is thus possible.

In some embodiments, it may further be provided to pattern the support surface in the first or second region to produce improved light coupling or light decoupling, respectively. In some aspects, a biosensor according to the proposed principle comprises a first optical element, for example in the form of a lens. This is arranged above the support surface above the first optical light guide element. In some examples, the optical element may also form a portion of the first optical fiber element adjacent the support surface. Such a first optical element is used, for example, to collect or collimate light beams that strike the support surface at a very small angle to the normal. This further increases the useful signal component. Similarly, an optical element is possible which is configured to refract light components incident at a very large angle,—i.e., an angle above or near the critical angle—away from the first optical fiber element by the additional first optical element, so that the DC signal component on the detector is further reduced.

In another aspect, a second optical element is provided that may be configured, in particular, as a lens. In such an embodiment, the lens can serve, for example, to collimate the light emitted by the emitter device and thus focus it into the deepest possible tissue layers and increase the penetration depth. This can increase a signal component that interacts with blood or hemoglobin in the deeper tissue layers and thus contributes to the useful signal component for the detector.

In some further aspects, the emitter device may be formed by a light emitting diode having a radiation pattern substantially corresponding to a Lambertian radiation pattern. In other aspects, vertically emitting lasers or edge emitting lasers may be provided. These produce a more directional light, such that deeper tissue layers and thus a stronger interaction with hemoglobin or blood can be achieved with these. Thus, in some aspects, the emitter device provided is a laser array, particularly a VCSEL. An edge-emitting laser is also suitable if, for example, it is either arranged at right angles within the housing, or emits light in the direction of the support surface via a reflector.

Depending on the desired application, in some aspects it is convenient to provide an emitter device that does not emit light of only a single wavelength or a few wavelengths, but rather generates light over a larger portion of the visible spectrum or even the invisible spectrum. Accordingly, in some aspects, an emitter device is disclosed that is configured to emit light in the green, red, infrared, or other suitable visible or invisible wavelength. Such light generation may be accomplished by a plurality of individual semiconductor devices but also by means of converter particles or other suitable means.

As mentioned above, other optical elements such as lenses, Fresnel lens filters or other elements may be provided in the beam path of the emitter device to select light of the appropriate wavelength for the measurement and radiate it onto the support surface and thus toward the human tissue.

Another aspect relates to a method of manufacturing a biosensor. The method comprises providing a housing having a housing wall and a housing bottom. An emitter device is disposed, particularly by adhesive bonding, to the housing bottom in a first region of the housing. Likewise, a detector device is arranged, in particular by adhesive bonding, in a second region of the housing. A first optical fiber element with a transparent material and a first refractive index is also arranged above the detector device, in particular by adhesive bonding. The system of detector device and light guide element can also be manufactured together in advance.

After insertion of the various elements, at least the second area is now filled with a potting material so that a contact surface is formed which is essentially flush with a surface of the first optical element. The potting material has a second refractive index that is smaller than the first refractive index. As a result, an interface is formed between the first optical fiber element and the potting material, at which incoming light is reflected depending on the angle.

In a further aspect, a second optical fiber element comprising a transparent material of a third refractive index is secured over the emitter device, in particular by bonding. The first region may likewise be filled with a potting material such that a bearing surface is formed which is substantially flush with a surface of the second optical element. Such potting material is also filled when no other second optical fiber element is provided. The potting material has a fourth refractive index, which is smaller than the third refractive index, so that an interface is formed between the second optical fiber element and the potting material, at which incoming light can be reflected depending on the angle.

The method thus creates optical light guides that can be used to influence the radiation properties of light and to remove DC components from tissue layers near the surface. In one aspect, the first and second optical light guide elements comprise the same material. Similarly, the potting material of the first and second regions may also be the same potting material. The method may implement the forms of light guide elements described above for the first and second optical light guide elements.

Similarly, in some aspects, an additional optical element may be disposed above the support surface above the first and/or second optical light guide element. Similarly, the first and or second optical light guide elements may also be implemented with an additional optical element configured to guide light. These can be lenses, collimators or other elements. In this respect, in addition to the optical light guide elements, in some aspects additional optical elements are provided over the emitter device and/or the detector device.

### BRIEF DESCRIPTION OF THE DRAWINGS

Further aspects and embodiments according to the proposed principle will become apparent with reference to the various embodiments and examples described in detail in connection with the accompanying drawings.

FIG. 1 shows a schematic diagram of a biosensor that can be used, for example, for PPG measurements;

FIG. 2 forms a schematic representation of a first embodiment to explain some aspects of the proposed principle;

FIGS. 3A to 3C each show a section of a biosensor to explain some aspects of the proposed principle;

FIG. 4 presents an embodiment example in its application to explain some aspects of the proposed principle;

FIG. 5 shows an embodiment example in its application to explain some aspects of the proposed principle;

FIGS. 6A and 6B represent sections of various aspects to explain the proposed principle;

FIG. 7 shows another embodiment of a biosensor illustrating some aspects of the proposed principle;

FIGS. 8A and 8B show various embodiments of a biosensor to explain some aspects of the proposed principle;

FIGS. 9A and 9B show various embodiments of another biosensor to explain some aspects of the proposed principle;

FIG. 10 shows a block diagram with essential aspects of signal generation and processing;

FIG. 11 shows another embodiment of a biosensor according to the proposed principle.

### DETAILED DESCRIPTION

The following embodiments and examples show various aspects and their combinations according to the proposed principle. The embodiments and examples are not always to scale. Likewise, various elements may be shown enlarged or reduced in size to highlight individual aspects. It will be understood that the individual aspects and features of the embodiments and examples shown in the figures may be readily combined without affecting the principle of the invention. Some aspects have a regular structure or shape. It should be noted that minor deviations from the ideal shape may occur in practice, but without contradicting the inventive idea.

In addition, the individual figures, features and aspects are not necessarily shown in the correct size, nor do the proportions between the individual elements have to be fundamentally correct. Some aspects and features are highlighted by showing them enlarged. However, terms such as “above”, “above”, “below”, “below”, “larger”, “smaller” and the like are correctly represented in relation to the elements in the figures. Thus, it is possible to derive such relationships between the elements based on the figures. However, the proposed principle is not limited herein, but different optoelectronic devices, with different size and also functionality can be used in the invention. In the embodiments, elements with the same or similar effects are shown with the same reference signs.

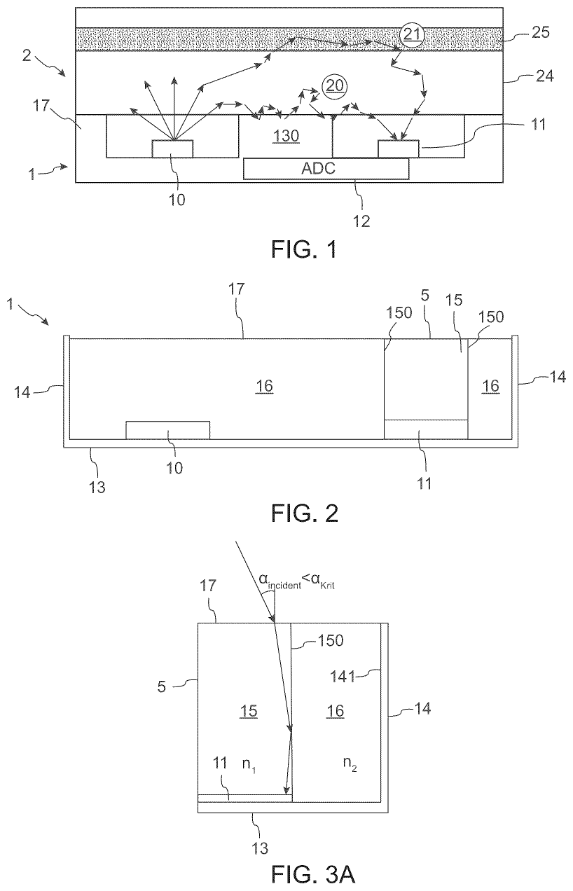

FIG. 1 shows the principle of a biosensor for measuring various vital parameters such as pulse in human tissue. The biosensor comprises a housing with a contact surface 17 that comes into contact with the human tissue 2. In use, the biosensor is pressed onto the skin with the contact surface 17. The biosensor includes a light emitter device 10 in the form of one or more light emitting diodes or other light generating components, and a photodetector 11. The photodetector and also the light emitter device are connected to a control and evaluation circuit 12. An opaque barrier 130 is also disposed between the light emitter device 10 and the photodetector 11.

In one operation of such a biosensor, the light emitter device generates light and emits it into human tissue. As shown in the embodiment of FIG. 1, the emitted light propagates along various optical paths, two of which are shown here as examples. In a first path 20, the light emitted by the light emitter device is reflected in different areas and then enters the photodetector. This reflection and scattering of light occurs in an area 24 of the tissue near the surface, through which blood does not flow. In this respect, this scattered portion does not contribute anything to a further measurement result and forms a DC portion and part of the noise, since this is relatively independent of the vital parameter being measured.

A second path 21, however, is located in deeper tissue layers and leads in particular via a vein or another blood stream 25 back to the detector 11. In the signal path shown here, the light in the human tissue interacts with the blood stream 25 or the hemoglobin contained therein. This interaction results in a modulation of the scattered light received in the detector, correlated to the heartbeat. The total signal received in the detector has, in addition to the unmodulated DC component, a component which has a fixed temporal relation to the pulse of the organism. The detector arrangement 11 detects the total signal and feeds it to a selected circuit.

In the following, the DC or DC signal portion is understood to be the portion of the scattered light received by the detector 11 which does not carry any information relevant for the measurement. These are, among other things, light components which originate from a direct crosstalk of light from the light emitter device into the detector, but also the light components of the signal path 20 and those of the path 21, which are nevertheless unmodulated. The useful signal is understood to be those parts of the light which interact in deeper tissue layers with the hemoglobin or the parameter to be measured and are thus altered by it in a measurable and specific manner.

The inventors now propose an improved arrangement of biosensor, in which the DC components are reduced, resulting in an improved signal-to-noise ratio. In conventional products with such a function, for example so-called smart watches, optoelectronic components are usually placed as emitters and the corresponding photodetectors are placed directly below a cover glass but at a relatively large distance from each other. The distance is necessary to reduce the stray light of the path 20 shown in FIG. 1 as much as possible so that the signal-to-noise ratio permits sufficiently good signal processing.

In addition, an optical barrier is incorporated between the photodetector 11 and the light emitter device 10 to further reduce crosstalk. The cover glass is also blackened in certain areas with an absorbent ink. In particular elaborate embodiments, the cover glass is completely penetrated by a barrier in the vertical direction so that it extends to the contact surface with human tissue. While the above approaches can result in practical and effective suppression of the DC component due to direct crosstalk between the emitter and detector, they require relatively large distances between the two units. This precludes certain applications that require the smallest possible design.

The inventors now propose an arrangement with the aid of which light leading to a DC component can also be effectively suppressed in the tissue layers near the surface. FIG. 2 shows a schematic embodiment of the proposed principle to explain the idea of the invention.

The biosensor according to FIG. 2 comprises a housing with a housing underside 13 and a housing wall 14 adjoining it. An emitter device 10 is arranged on the housing underside in a first area and a detector device 11 is arranged in a second area separated therefrom. These can, for example, be glued to the underside of the housing 13 or otherwise fastened to it and are in electrical contact with a control and evaluation circuit not shown here. In addition, the housing is filled with a transparent potting material 16, the surface of which also forms the contact surface 17 of the biosensor. The contact surface is in contact with the skin either directly or also via an additional optional cover glass.

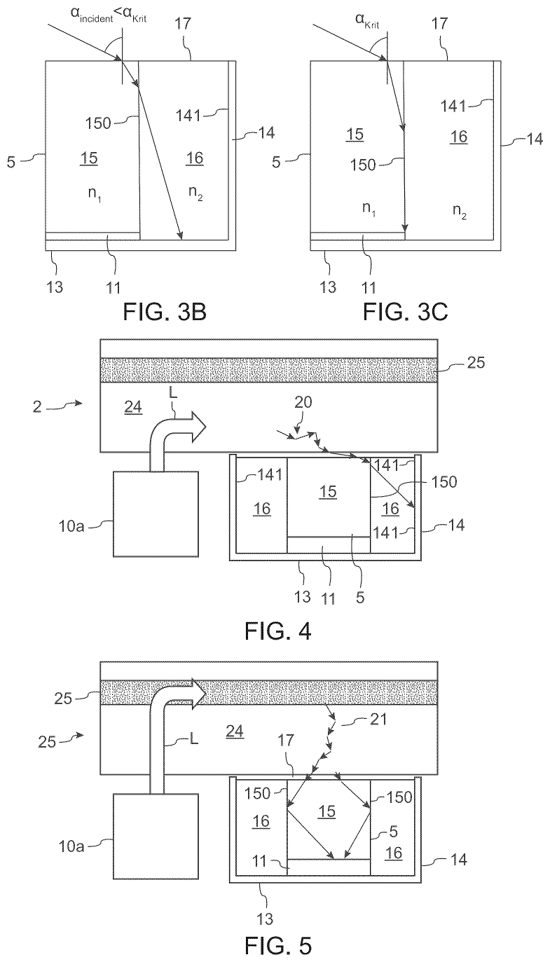

According to the invention, a first optical fiber element 5 is now provided above the detector 11. The first optical fiber element 5 comprises a highly refractive transparent material 15, which in the embodiment example of FIG. 2 extends from the surface of the detector device to the support surface 17. Material 15 comprises a refractive index n1, and is surrounded by a second transparent material 16. This, however, comprises a refractive index n2 which is smaller than the first refractive index n1. When this condition is met, the optical element 5 above the detector device acts as a light guide with an aperture characteristic thereof, which is explained in more detail in FIGS. 3A to 3C.

FIGS. 3A to 3C show a section of the second area of the biosensor with the photodetector 11, the first optical fiber element 15 and the surrounding material 16. As shown in FIGS. 3A to 3C, the transparent material 15 of the optical fiber element with the refractive index n1 is arranged above the detector. An interface 150 is formed between the optical fiber element 15 and the material 16 surrounding the optical fiber element with refractive index n2, which is shown here extending perpendicularly from the detector surface to the support surface 17.

The different refractive indices result in a reflection angle at the interface 150, so that the interface 150 totally reflects light when it is incident up to a certain angle. If the light is followed back along the optical path, the angle at which the light enters the light guide element can also be determined for the impact of the light on the contact surface.

Light thus strikes the surface of the light guide element, which forms part of the contact surface, at a certain angle to the perpendicular. There, it is refracted in the direction towards the perpendicular and can reach the interface depending on the initial angle of incidence. The resulting angular relationships allow to determine a critical angle αₖᵣᵢₜ until, moreover, a light impinging on the support surface 17 is guided inside the light guide element 5. The possible cases for this are shown in FIGS. 3A to 3C.

This results in a critical angle αₖᵣᵢₜ, which is essentially proportional to an arc sine of the root of the difference of the respective squared refractive indices. It results for the numerical aperture NA:

$$N A = sin ( {α}_{krit} ) = \sqrt{{n}_{1}^{2} - {n}_{2}^{2}}$$

Here, n1 and n2 are the respective refractive indices of the materials of the optical waveguide and the material surrounding the optical waveguide. Now, if the angle of incidence α_{incidence} is smaller than the predetermined critical angle αₖᵣᵢₜ, which results from the difference of the respective refractive indices, the rays falling on the optical waveguide 5 are totally reflected at the interface 150 and thus guided to the photodetector. This case is shown in FIG. 3A.

If the angle of incidence α_{incidence} of the light rays as shown in FIG. 3B is greater than the predetermined angle αₖᵣᵢₜ, the light rays are refracted out of the first optical fiber element 5 when they strike the interface 150 and thus reach the surrounding material 16 and the wall of the housing, where they are absorbed. At the angle αₖᵣᵢₜ shown in Figure C, the angle of incidence is the limiting angle above which incident light will not reach the photodetector.

By a correspondingly suitable choice of the refractive indices n1 and n2, the appropriate critical or predetermined angle αₖᵣᵢₜ for guiding light through the first optical fiber element 5 can thus be set.

In doing so, the inventors take advantage of the fact that scattered light, which reaches the entrance of the first optical fiber element from the deeper tissue layers and thus interspersed with hemoglobin, has a relatively small angle to the perpendicular. Accordingly, the angle of incidence α_{incidence} is often smaller than the predetermined angle αₖᵣᵢₜ, so that light impinging on the support surface 17 is guided by the optical waveguide toward the detector.

On the other hand, light components from the tissue layers close to the surface are usually scattered in such a way that their angle of incidence α_{incidence} onto the contact surface 17 and thus the first optical fiber element is greater than the predetermined or critical angle αₖᵣᵢₜ. This is shown, for example, in FIG. 3B. Accordingly, such light components are refracted out of the light guide element and no longer enter the photodetector. The signal-to-noise ratio, i.e. the proportion of the useful signal with an angle smaller than the critical angle, thus increases in the entire detected signal.

In this context, FIGS. 4 and 5 again illustrate the principle according to the invention by means of two scattered light signals along paths 20 and 21, respectively. The emitter device 10a is designed here as a separate element, and the detector element is embedded in a housing with a housing wall 14. Above the photodetector 11, in both FIGS. 4 and 5, the first optical fiber element 5 with its high refractive index transparent material 15 is applied and surrounded by the casting material 16 with a low refractive index.

FIG. 4 shows the example of the light component from near-surface tissue layers, for example, along the path 20. The light emitted from the emitter device 10a enters the tissue and is deflected by various scattering and interaction processes toward the support surface 17 for the biosensor. However, due to the near-surface scattering, the signal path as shown causes the scattered light components to strike the surface of the optical element 5 at a relatively large angle to the perpendicular of the support surface and the first optical element 5.

Thus, although they are coupled into the optical element, they are refracted into the transparent material surrounding the optical fiber element 5 when they strike the interface 150 and thus reach the walls of the housing 14 where they are absorbed. A light component refracted out of the optical fiber element 5 in this way thus no longer contributes to the overall measurement result and thus reduces the DC or noise component of the photodetector.

FIG. 5, on the other hand, shows a light path 21 which is formed, for example, by an interaction of a light component with deeper tissue layers 25 containing hemoglobin. Here, as in the previous example, light is generated by the emitter unit 10a, whereby a certain light component now penetrates into deeper tissue layers and interacts there with hemoglobin and the blood in the area 25. The interacting portion is again guided toward the photodetector by various scattering processes and now strikes the support surface 17 and the surface of the first optical fiber element at a relatively small angle. The small angle is below the critical angle αₖᵣᵢₜ caused by the various refractive indices, so that the light rays entering the optical fiber element 5 are totally reflected at the interface 150 and guided onto the detector 11.

The specified or critical angle, as already mentioned, depends on the differences in the refractive indices. Material suitable for the optical light guide element, for example silicone, or polycarbonate has a refractive index of 1.6 or higher. Meanwhile, the transparent encapsulant 16 surrounding the optical fiber element 5 is made of a low refractive material with a refractive index of 1.5 or even lower. Suitable materials in this case would be plastics. In a further example, the refractive index for n2 is less than 1.5, while the refractive index for n1 is greater than 1.5.

With these two exemplary refractive indices, the given angle αₖᵣᵢₜ for total reflection would be, for example, αₖᵣᵢₜ=50.7°. Accordingly, rays that reach the surface of the optical fiber element from deeper tissue layers and at the same time have a Lambertian distribution with a maximum perpendicular to the contact surface would be guided to a considerable extent to the detector by total reflection in the first optical fiber element and contribute to signal generation there. As shown in the example in FIG. 4, however, light components are scattered at the upper tissue layers in such a way that they have a different distribution, in particular a distribution with maxima at angles greater than the critical angle. Thus, these light components are largely refracted out of the optical fiber element 5 into the transparent potting compound 16 and subsequently absorbed by the side walls 14 or the back of the housing 13. As a result, the overall proportion of the detector's signal modulated by the hemoglobin increases and the so-called modulation depth increases.

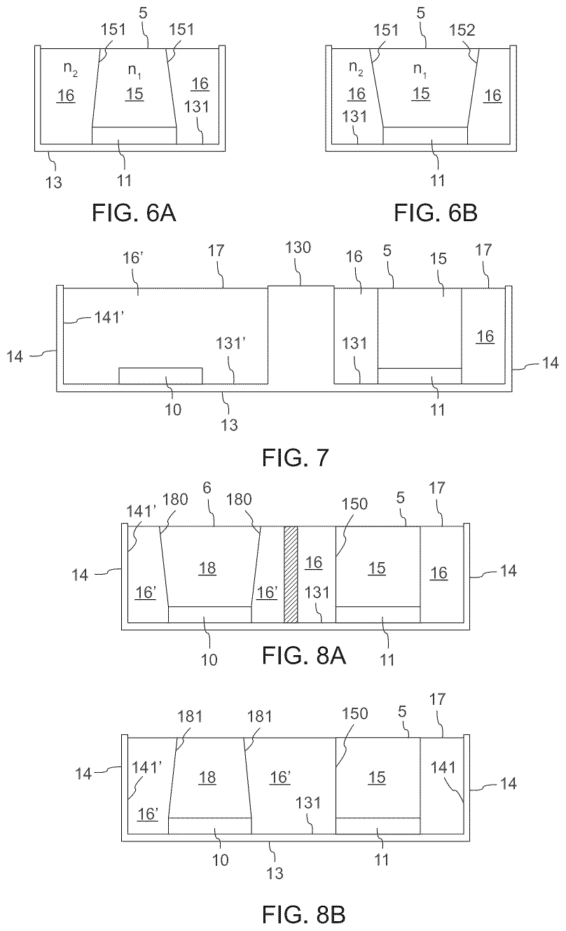

FIGS. 6A and 6B show a further embodiment of the second region of a biosensor with a photodetector 11 for detecting the light components scattered and reflected by tissue layers. In contrast to the previous examples, here the light guiding element 5 has an additional shape optimization in which further aspects and parameters can be adjusted.

In part FIG. 6A, the first light conducting element is designed as a truncated pyramid or a truncated cone. In cross-sectional view, therefore, it shows a cross-section tapering towards the support surface 17. In other words, the cross-section and the diameter of the material 15 of the first optical fiber element 5 become smaller in the direction towards the supporting surface. As a result, light coupled into the first optical fiber element and impinging on the interface 151 has a smaller angle between the impinging light and the interface. In other words, this also reflects light that would already be refracted into the surrounding material 16 if the interface were perpendicular. Accordingly, the critical angle increases compared to a perpendicular interface, so that altogether more components enter the detector 11. However, the new shape also reduces the entrance area in the contact surface 17, so that a small part of the total signal, i.e. the useful signal as well as the unusable DC components, is lost again.

FIG. 6B, on the other hand, shows a design in which the cross-section of the first optical fiber element 5 tapers from the support surface 17 in the direction of the detector surface 11. Thus, the first optical fiber element is designed as an inverted pyramid structure or as a funnel structure. Due to the larger entrance window, more useful but also DC components are received. At the same time, the critical angle for total reflection is reduced due to the slanted interface 152, so that more signal components are refracted out of the first optical fiber element into the surrounding medium.

The embodiments of FIGS. 6 and 6B thus show shapes in which an increase in the coupled light signal components simultaneously leads to increased coupling out at the interface and vice versa. A preferred shape of the first optical element can be selected by appropriate simulations and by a suitable combination of materials between the optical fiber element and the transparent potting material, the length of the optical element and the distance between the emitter and detector.

FIG. 7 shows an embodiment of a biosensor in which a housing is provided with a first region and a second region arranged adjacent thereto. First and second areas are separated from each other by an absorbing wall 130. A transparent material 16, or 16′, is introduced into both regions to surround the detector device 11 and the emitter device 10, respectively. The two potting materials may be of the same material, but may also be made of different transparent materials depending on the application and need. In the first area above the detector device 11, a first optical fiber element 5 is now also provided, the material 15 of which has a significantly higher refractive index than the surrounding material.

The wall 130 provided between the first and second regions reduces direct crosstalk of light components from the emitter device 10 into the detector 11. In one embodiment, the wall 130 may also extend slightly beyond the support surface 17 in the first and second regions.

FIGS. 8A and 8B show further embodiments of a biosensor with some aspects of the proposed principle In these, the biosensor comprises a housing with a circumferential side wall 14 and a bottom surface 13, in each of which an emitter device 10 is bonded into a first region and a detector device 11 is bonded into a second region. Optical light guide elements 5 and 6 are now arranged above both the detector device and the emitter device, the materials 15 and 18 of which have a significantly larger refractive index than the surrounding material.

In subfigure 8A, a separating wall 130 is also provided between the first and second regions. The surface of this wall is respectively made with absorbent particles similar to the bottom 13 and the side wall 141, so as to prevent reflection of light from these walls. This applies to both the first area with the emitter device 10 and the second area with the detector device 11.

In subfigure 8A, the first optical fiber element 5 has vertical side walls and thus a vertical boundary surface 150. In contrast, the second optical element 6 with its material 18 has an oblique boundary surface 180 and in particular a funnel-shaped course. Light emitted by the emitter device which falls on the boundary surface 180 is thus directed more parallel and emitted directly upwards. This makes it possible to achieve more directional radiation into the fabric resting on the surface.

FIG. 8B, on the other hand, shows a pyramid-shaped structure for the second optical fiber element 6, i.e. the cross-section of the second optical fiber element 6 tapers in the direction of the support surface 17. This also results in obliquely extending interfaces 181 between the material 18 and the surrounding transparent potting material 16′

The additional optical light guide elements for the emitter device shown here can be integrated into the housing, for example in the form of a pyramid or funnel shown here. In addition, further optics can also be applied above the emitter device in order to collimate the light emitted by it and to bring more light into the tissue resting on the support surface

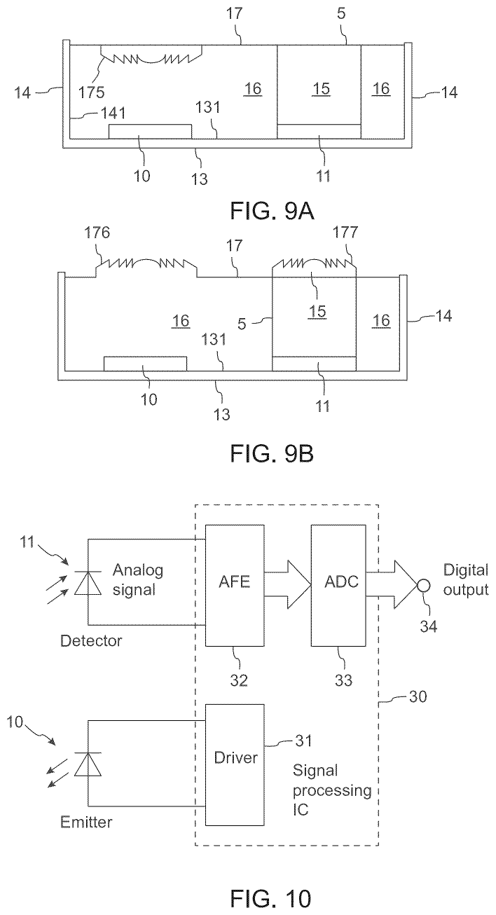

FIGS. 9A, and 9B show corresponding embodiments and can be readily combined with the preceding embodiments. In partial FIG. 9A, an additional optical element 175 in the form of a lens is arranged in the beam path of the first area above the emitter device 10. This is incorporated in the potting material 16 and is flush with the contact surface 17.

In contrast, in subfigure 9B, an additional optical element 176 is arranged in the beam path of the emitter device 10 above the support surface 17. A corresponding optical element 177 is provided above the entrance window of the first optical fiber element 5. Thus, the supporting surface 17 is no longer flat, but structured by the additional optical elements 176 and 177. An additional optical element 176 in the beam path of the emitter device serves to align the light emitted by the emitter device and to largely reduce scattering, especially in tissue layers close to the surface. This further reduces a DC component coupling into the first optical light guide element. Similarly, the second optical element 177 also acts in a manner that, depending on the application and design, can be configured to further favorably influence the critical angle in terms of the application.

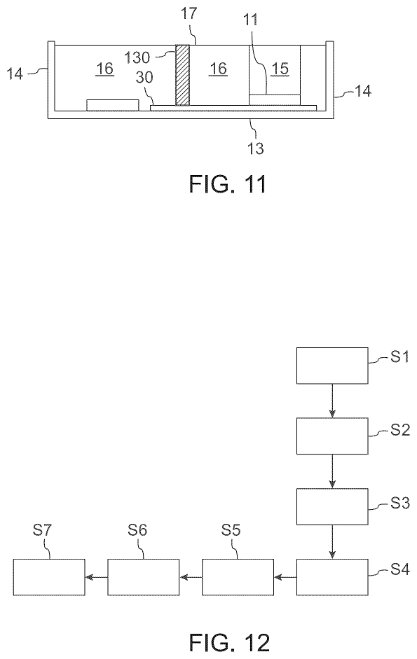

FIG. 11 shows an embodiment of a biosensor in which an integrated circuit 18 is implemented within the housing. The integrated circuit 30 is attached to the bottom 13 of the housing with an adhesive, and the detector arrangement 11 is provided on a portion of the circuit. A wall 130 separates the first area from the second area within the housing, so that direct crosstalk is largely avoided.

In the preceding examples, the emitter devices 10 can be implemented by various optoelectronic devices. In one simple but effective embodiment, the emitter device 10 is implemented using one or more light emitting diodes, wherein these light emitting diodes are configured to emit light of different colors. In another embodiment, the light emitting units are implemented as laser devices. Lasers have the property that they emit a significantly more directional radiation compared to conventional light emitting diodes, which in particular does not have a Lambertian radiation characteristic.

As a result, laser devices introduce a larger proportion of light into deeper tissue layers. The directional radiation, which can also be amplified by additional optical elements, may reduce scattering so that a larger proportion of light can interact with blood capillaries or hemoglobin. In some embodiments, the lasers may be vertical cavity emitting lasers (VCSELs). However, it is also possible to provide an edge-emitting laser and to glue it vertically in the housing, for example on an auxiliary carrier.

A third possibility is to use an edge-emitting laser as a “side-loader” in order to radiate the light emitted by it in the direction of the support surface using a suitable reflector or mirror. Converters or scattering particles can also be provided in the potting material to achieve a desired radiation pattern even without additional optics. Converter particles are suitable for converting the light emitted by the laser devices into light of a second wavelength and then radiating it into the tissue layers. This broadens the spectrum of the emitted light, which may be necessary in some applications.

FIG. 10 shows a block diagram with the essential elements for signal processing of a biosensor according to the proposed principle.

Signal processing takes place in a control and evaluation circuit 30, which is arranged inside the housing as shown in FIG. 11. The control and evaluation circuit 30 comprises a driver circuit 31 which leads to the emitter device 10 for its supply. An analog receiving unit 32, which may include filters, amplifiers and similar components, is connected to the photodetector 11. On the output side, the analog receiving unit is connected to an analog-to-digital converter 33 which converts the signal received from the photodetector 11 and processed in the analog front end 32 into a digital word and supplies it to a digital output 34.

A major requirement for the analog-to-digital converter 33 arises from the dynamic range and sensitivity, i.e., resolution and noise ratio. As mentioned above, the useful signal component in conventional biosensors is quite small compared to the DC component. Thus, it is either necessary to design the AD converter with a very large dynamic range in order to still capture the modulation of the useful signal with a good resolution. Furthermore, the analog front end includes the possibility to subtract an offset as a DC component from the input signal before this is fed to the actual ADC. This provides the possibility to amplify the cleaned input signal analog without exceeding the input dynamic range of the ADC.

This may be possible in some applications, but the DC component fluctuates due to the change in the distance between biosensor and tissue surface due to motion or even individual operation is relatively strong. This leads to the fact that the correction of the input signal would have to be done iteratively and in turn with an increased resolution, which significantly increases the requirement for such a circuit-based solution and requires several quantization stages for the input compensation. This in turn limits the gain of the input signal.

With the measures presented here, the DC component in the overall signal is reduced by the optics as such. This significantly reduces the requirements for the correction of the input signal, since the fluctuations of the measuring path have a much smaller effect on the DC component, thus simplifying correction of this component, both in terms of speed and resolution.

Another significant advantage results from the lower signal level of the optical input power with the same power coupled into the skin, since the DC component is reduced by the optics and the measured quantity is almost identical. Since the input dynamic range of the measurement path is limited, in a conventional setup the maximum possible coupled light power is defined by the DC component. By reducing the DC component, the light power can be increased. This leads to an increase in the signal-to-noise ratio in relation to the measured variable.

FIG. 12 illustrates a method of manufacturing a biosensor. In step S1, a housing with a housing wall and a housing base is provided. The housing forms a cavity in which the sensor elements are inserted in steps S2 and S3. These are, firstly, in step S2, an emitter device which is bonded to the housing base in a first region. In a second area separated therefrom, a detector device is glued to the building floor in step S3.

Alternatively, a control and evaluation circuit that includes the photodetector can be used. In addition, the first and second areas can be separated by a wall, whereby the wall can form part of the housing but can also be added subsequently.

In step S4, a first optical fiber element is bonded over the detector device using a transparent material. The first optical fiber element has a first refractive index. Similarly, in step S5, a second optical fiber element with a transparent material of a third refractive index is bonded over the emitter device. In this example, the optical light guide elements are separately attached to the emitter device or detector device. However, the systems can also be fabricated together beforehand, so that steps S4 and/or S5 occur before the emitter device and the detector device are glued into the housing. The two light guide elements comprise the same shape, but may also have different shapes.

After insertion of the different elements, the first and second regions are now filled with a potting material in step S6 so that a contact surface is formed which is essentially flush with a surface of the first and second optical elements. The potting material has a refractive index that is smaller than the refractive indices of the materials of the two optical light guide elements. As a result, an interface is formed between the first optical fiber element and the potting material, at which incoming light is reflected depending on the angle.

The method thus creates optical light guides that are used to collimate light, on the one hand, and to remove DC components from tissue layers near the surface, on the other hand. In one aspect, the first and second optical light guide elements have the same material. Similarly, the potting material of the first and second regions may also be the same potting material.

Likewise, in an additional step S7, additional optical elements are arranged above the support surface above the first and/or second optical light guide element. As already explained, these are designed as lenses to collimate the light and increase the proportion of the useful signal in the overall signal measured by the detector.

### REFERENCE LIST

5 first optical light guide element
6 second optical light guide element
10 Emitter device
11 Detector device
15 Material
16 Potting material
18 Material
20, 21 signal paths
24, 25 Fabric
30 Control and monitoring circuit
131, 141 Surface
150 Boundary
151, 152 Boundary surface
175 optical element, lens
176 optical element, lens
177 optical element, lens
180, 181 Interface

1. Optoelectronic biosensor comprising
a housing with a transparent support surface;
an emitter device in a first region of the housing adapted to generate and emit light toward the support surface;
a detector device in a second region of the housing;
a first optical fiber element disposed between the detector device and the support surface, the first optical fiber element being configured to guide light incident on the support surface at an angle less than a predetermined angle to a normal to the detector device, the predetermined angle depending at least on a difference in refractive indices at an interface between the optical fiber element and a material surrounding the optical light guide element;
wherein a material of the first region surrounding the emitter device is the same as the material surrounding the first optical fiber element.

2. The optoelectronic biosensor of claim 1, wherein a material of the first optical fiber element has comprises a higher refractive index than the material surrounding the first optical fiber element.

3. The optoelectronic biosensor according to claim 1, wherein the predetermined angle is proportional to an arc sine of a root of the difference of the two respective squared refractive indices.

4. The optoelectronic biosensor according to claim 1, wherein at least the second region is filled with the material surrounding the first optical fiber element up to a height of the support surface.

5. The optoelectronic biosensor according to claim 1, wherein the material surrounding the first optical fiber element is transparent.

6. The optoelectronic biosensor according to claim 1, wherein a surface of the housing facing the second region is configured to be absorbent of the light emitted by the emitter device.

7. The optoelectronic biosensor according to claim 1, wherein the material surrounding the first optical fiber element comprises at least one of the following materials:
low refractive plastic;
Epoxy resin;
transparent material with a refractive index less than 1.5;

8. The optoelectronic biosensor according to claim 1, further comprising:
a second optical fiber element disposed between the emitter device and the support surface for guiding light emitted from the emitter device.

9. The optoelectronic biosensor according to claim 1, wherein the first optical fiber element and/or the second optical fiber element comprises at least one of the following materials:
Silicon;
Polycarbonates;
Glass with a refractive index greater than 1.5.

10. The optoelectronic biosensor according to claim 1, wherein the first optical fiber element and/or the second optical fiber element comprises a cross-section tapering towards the support surface.

11. The optoelectronic biosensor according to claim 1, wherein the first optical fiber element and/or the second optical fiber element comprises a cross-section that increases towards the support surface.

12. The optoelectronic biosensor according to claim 1, wherein the first region of the housing is separated from the second region of the housing by an absorbent region extending from a bottom of the housing to the support surface.

13. The optoelectronic biosensor according to claim 1, wherein the support surface is structured in the first and/or second region.

14. The optoelectronic biosensor according to claim 1, further comprising:
a first optical element, in particular a lens, which is arranged above the supporting surface above the first optical light guide element, or
which forms a part of the first optical fiber element adjacent to the support surface.

15. The optoelectronic biosensor according to claim 1, further comprising:
a second optical element, in particular a lens,
which is arranged in a beam path of the emitter unit, in particular above the supporting surface;
or which forms a part of the second optical fiber element adjacent to the supporting surface.

16. The optoelectronic biosensor according to claim 1, wherein the emitter device is formed by at least one of the following:
a light emitting diode with an essentially Lambertian radiation pattern;
a laser device, in particular a VCSEL laser, which generates a substantially directional light;
an edge-emitting laser with an optional deflection device.

17. The optoelectronic biosensor according to claim 1, wherein the emitter unit is configured to generate and emit light of different wavelengths.

18. The optoelectronic biosensor according to claim 1, further comprising a control circuit disposed in the second region of the housing and on which the detector means is optionally placed.

19. A method of making a biosensor comprising the steps of:
providing a housing having a housing wall and a housing bottom;
arranging, in particular by gluing, an emitter device the bottom of the housing in a first region of the housing;
arranging, in particular by bonding, a detector device in a second region of the housing;
arranging, in particular by bonding, a first optical fiber element with a transparent material of a first refractive index over the detector device;
filling at least the second region with a potting material so as to form a bearing surface which is substantially flush with a surface of the first optical element;
wherein the potting material comprises a second refractive index which is smaller than the first refractive index, so that an interface is formed between the first optical fiber element and the potting material, at which interface incident light can be reflected as a function of angle.

20. The method of claim 19, further comprising the step of:
arranging, in particular by bonding, a second optical fiber element with a transparent material of a third refractive index over the emitter device;
filling at least the first area with a potting material so that a support surface is formed which is substantially flush with a surface of the second optical element;
wherein the potting material comprises a fourth refractive index which is smaller than the third refractive index so that an interface is formed between the second optical fiber element and the potting material, at which incident light can be reflected in an angle-dependent manner.

21. The method according to claim 19, wherein the first and second optical light guide elements comprise the same material; and/or the potting material of the first and second regions is the same potting material.

22. The method according to claim 19, wherein the first optical fiber element and/or the second optical fiber element comprises a cross-section tapering toward the support surface.

23. The method according to claim 19, wherein the first optical fiber element and/or the second optical fiber element comprises a cross-section that increases toward the support surface.

24. The method according to claim 19, further comprising the step of:
arranging a first optical element disposed above the support surface over the first optical light guide element; or
forming a first optical fiber element with an additional first optical element designed for guiding light.

25. The method according to claim 19, further comprising the step of:
arranging a second optical element in a beam path of the emitter unit, in particular above the support surface formed by the potting material; or
forming a second optical fiber element with an additional second optical element designed for guiding light.
